(19) United States
(12) Patent Application Publication
(10) Pub. No.: US 2020/0158729 A1
(43) Pub. Date: May 21, 2020

(54) SIMULTANEOUS IN VITRO ANALYSIS OF VACCINE POTENCY AND TOXIN CONCENTRATION

(71) Applicant: INDEVR, INC., Boulder, CO (US)

(72) Inventors: Kathy ROWLEN, Boulder, CO (US); Rose BYRNE-NASH, Boulder, CO (US); Laura KUCK, Boulder, CO (US); Erica DAWSON, Boulder, CO (US)

(73) Assignee: INDEVR, INC., Boulder, CO (US)

(21) Appl. No.: 16/615,372

(22) PCT Filed: Jun. 6, 2018
(86) PCT No.: PCT/US2018/036250

§ 371 Date: Nov. 20, 2019

(51) Int. Cl. G01N 33/569; A61K 39/39; C07K 16/08; A61K 39/12; G01N 33/533; A61K 39/02; C07K 16/12; C40B 30/04; A61K 39/00

(52) U.S. Cl. CPC G01N 33/56983; A61K 39/39; C07K 16/08; A61K 39/12; G01N 33/56911; G01N 2400/50; A61K 39/02; C07K 16/12; C40B 30/04; A61K 2039/555; G01N 33/533

(57) ABSTRACT

Provided herein are systems and methods to determine vaccine potency and toxin concentration. The provided systems and methods may be multiplexed to determine potency and toxin concentration simultaneously. The systems and methods may be microarray based, providing accurate results while reducing the amount of testing time required compared to current potency and toxin concentration tests which often require the use of animal subjects or expensive test materials. Further, the provided systems and methods may detect desired antigens, endotoxins and exotoxins.

### CROSS-REFERENCE TO RELATED APPLICATIONS

This application claims the benefit of priority to U.S. Provisional Patent Application No. 62/515,958 filed on Jun. 6, 2017, which is specifically incorporated by reference to the extent not inconsistent herewith.

### STATEMENT REGARDING FEDERALLY SPONSORED RESEARCH OR DEVELOPMENT

This invention was made with government support under Grant number R44A1102318 by the National Institute of Health (NIH). The government has certain rights in the invention.

### BACKGROUND

Vaccines play a key role in modern preventative medicine and are among the most effective, administered, and thus highest selling, pharmaceutical products. In determining vaccine potency, testing is performed to demonstrate the capability of the product to confer protective immunity and to ensure its stability over typical storage time periods. Currently expensive and complex in vivo animal-based assays are often utilized in potency testing. In addition, combination vaccines pose a particular challenge in that potency must be established for each component, often requiring a completely different methodology for each component. As such, it is highly desirable to develop suitable in vitro potency assays, particularly for combination vaccines for which the same general method can be applied to each component. According to the World Health Organization ICH guidelines, critical assay parameters for potency tests include accuracy, precision, linearity, range, specificity, stability indication, and robustness.

Combination vaccines, such as Pediarix, require expensive and complex in vivo potency methods for each of the vaccine components that make up the vaccine. For example, Pediarix has 9 components. Testing each component via a different method is expensive and time consuming. Additionally, several units of measurement are reported for the final formulation.

In addition, vaccine toxicity remains a key concern and hindrance to vaccine manufacturing. Toxins present in the vaccine production process, especially in later stages, tend to be in present at low concentrations but may still be harmful to patients. While significantly more guidance for the pharmaceutical industry exists for toxin concentration testing as compared to potency testing, current biological methods for toxin testing are time consuming and expensive.

Endotoxins, which are generally lipopolysaccharide complexes present in bacterial cell walls and can be liberated upon cell death, are common in vaccines and can be harmful to patients. Vaccine products that are nonconforming due to the presence of endotoxins may need to be destroyed to avoid potential for harm to patients. Typically, tests for endotoxins rely on blood from horseshoe crabs which contain amebocytes that coagulate around endotoxins. While this technique is sensitive, allowing for detection of some endotoxins to the part per trillion level, it is time consuming, expensive and results in the capture and death of hundreds of thousands of crabs per year.

Some vaccines may also contain undesired exotoxins, which are secreted directly from bacteria as part of their metabolic process and can be present during the vaccine manufacturing process. In some cases, multiple proteins are secreted by one or more types of bacteria, which then combine to generate an exotoxin. In these cases, it is important to also detect precursor proteins as well as exotoxins to ensure that toxicity does not later increase due to later combinations of precursors. For example, Anthrax Vaccine Absorbed (AVA) represents a vaccine in which secreted proteins combine to form an exotoxin. Specifically, three proteins may be present in AVA: Protective Antigen (PA), Lethal Factor (LF) and Edema Factor (EF). While Protective Antigen is necessary to generate an immune response in the patient, if it combines with either Lethal Factor or Edema Factor it becomes a potentially lethal exotoxin. Thus, vaccine manufactures have a goal of attaining a high concentration of Protective Antigen while minimizing Lethal Factor and Edema Factor.

It can be seen from the foregoing that there remains a need in the art to quickly and accurately determine the presence and/or concentrations of antigens (and thus vaccine potency) and possible toxin concentrations for both endotoxins and exotoxins in vaccines. Further, in vitro assays may provide the ability to determine potency for multiple vaccine components and toxin concentrations simultaneously, allowing for decreased testing costs, reduced vaccine production time, and more efficacious vaccines.

### SUMMARY

Provided herein are systems and methods to determine vaccine potency and toxin concentration. The provided systems and methods may be multiplexed to determine potency and toxin concentration simultaneously. The simultaneous characterization and/or quantification of both vaccine potency and toxin concentration in one assay has a number of fundamental advantages. For example, simultaneous testing reduces costs and test time, especially when compared to animal based testing methods. Further, the reduced testing time and costs may allow producers to test vaccine samples during various stages of the production process, allowing for recognition of problematic batches earlier and optimization of process steps due to greater insight into process results. Further, the ability to rapidly test vaccines increases vaccine safety and potentially reduces the risk of side effects due to toxins while ensuring vaccines are effective.

Any of the systems and methods provided herein may be microarray-based, and therefore multiplexed, providing accurate results while reducing the amount of testing time required compared to current potency and toxin concentration tests which often require the use of animal subjects or expensive test materials. Further, the provided systems and methods may detect desired antigens, endotoxins and exotoxins.

The method may be for quantifying vaccine potency and toxicity, such as by the steps of: i) providing a substrate having at least one antigen capture agent and at least one toxin capture agent; ii) contacting the substrate with a vaccine sample to form a plurality of bound complexes comprising: a) the antigen capture agent and an immunoactive antigen bound thereto; and b) the toxin capture agent and a toxin bound thereto; determining an immunoactive antigen concentration and a toxin concentration from the plurality of bound complexes, thereby simultaneously quantifying the vaccine potency and toxin concentration.

Any of the methods may be automated, for example, using automated microarray testing systems. Automated testing methods can decrease the required testing time and testing costs. Any of the methods may further comprise: i) generating a plurality of signals from the plurality of bound complexes by contacting the plurality of bound complexes with one or more label agents; ii) quantifying the plurality of signals to generate a plurality of quantified signals; iii) providing an antigen calibration curve that defines a relationship between the quantified signals with immunoactive antigen concentration; iv) providing a toxin calibration curve that defines a relationship between the quantified signals with toxin concentration; and v) calculating an immunoactive antigen concentration and a toxin concentration from the plurality of quantified signals, the antigen calibration curve and the toxin calibration curve.

The label agents may comprise fluorescently-labeled antibodies. In embodiments, the one or more label agents comprise an antigen label agent configured to specifically bind to the immunoactive antigen and a toxin label agent configured to specifically bind to the toxin. In embodiments, the step of generating a plurality of signals further comprises imaging the plurality of bound complexes to generate an image, and analyzing the image to generate the plurality of quantified signals.

The antigen calibration curve may be described as having a linear dynamic range, such as a range greater than or equal to 1 log, greater than or equal to 1.1 log, greater than or equal to 1.25 log, or, optionally, greater than or equal to 1.5 log; optionally the linear dynamic range may be described as having an upper limit of 3 log or 2 log. The toxin calibration curve may be described as having a linear dynamic range, such as a range greater than or equal to 1 log, greater than or equal to 1.1 log, greater than or equal to 1.25 log, or, optionally, greater than or equal to 1.5 log; optionally the linear dynamic range may be described as having an upper limit of 3 log or 2 log.

In an embodiment, the antigen capture agent and the toxin capture agent are provided in a microarray. In an embodiment, the substrate supports a plurality of microarrays. In embodiments, for example, the plurality of microarrays are each provided in a separate well. In an embodiment, wherein the antigen capture agents and/or the toxin capture agents comprise a panel of monoclonal antibodies. In embodiments, for example, the method has a run time of less than or equal to 3 hours, less than or equal to 2.5 hours, or optionally, less than or equal to 2 hours. Run-time as described above may exclude sample preparation, for example, the time required to solubilize any adjuvants.

The provided systems and methods may detect and quantify all antigens within a monovalent (containing a single strain of a single antigen), multivalent (containing two or more strains or serotypes of the same antigen), or combination vaccine (containing multiple antigens from multiple infectious agents, or multiple strains of infectious agents causing the same disease). Determining the potency of each component within a combination vaccine is labor, assay, and animal intensive. Each component of a combination vaccine must be tested, often separately, and often report very different units of measurement. The provided systems and methods are assay based, allow for rapid potency determination of all or some components of a combination vaccine in one, animal-free potency in vitro potency assay that reports a single unit of measurement, μg/mL immunogenic component. In some embodiments, vaccine potency may be quantified simultaneously with endotoxin or exotoxin using multiplex assays or microarrays.

The provided systems and methods may detect endotoxin, or toxins present in bacterial cell walls which may be released upon cell death. Endotoxins are common to many vaccine and vaccine types, while current methods exist (namely use of horseshoe crab blood as an adjuvant) to both detect and remove endotoxins from vaccines during production, current methods are expensive (horseshoe crab blood costs approximately $15,000/L), time consuming and result in animal deaths. The provided systems and methods are assay based (in vitro, as opposed to in vivo), allowing for rapid endotoxin detection and quantification without animal death or expensive reagents. Further, in some embodiments, endotoxins may be quantified simultaneously with desired vaccine antigen using multiplex assays or microarrays. In this manner, the level of contamination is quantified, with the appropriate resulting action dependent on the toxin level detected, ranging from less drastic low level of contamination that does not require immediate action or justifies enhanced future monitoring and/or assessment, to more drastic production shutdown and destruction of material.

In embodiments, for example, at least one toxin capture agent is configured to selectively bind to the toxin that is an endotoxin. In an embodiment, the endotoxin is a lipopolysaccharide generated in a bacterial cell wall and may be present in intermediate steps in the development of the vaccine sample. In embodiments, the endotoxin is a lipopolysaccharide from a gram negative bacterium, for example, Escherichia coli, Pseudomonas sp., Salmonella typhosa, Salmonella enterica, Klebsiella pneumoniae, Serratia marcescens, and Enterobacter sp.

As endotoxins are common in many vaccines, the provided systems and methods are useful for vaccines used to inoculate against a wide range of viral and bacterial infections. Accordingly, the systems and methods are applicable to a wide range of vaccines. Examples of vaccines are those comprising antigen against a disease, including but not limited to those selected from the group consisting of: influenza virus infection, diphtheria, human papillomavirus (HPV) infection, tetanus, pertussis, poliomyelitis, hepatitis, shingles, varicella, rotavirus infection (gastroenteritis), pneumonia, meningitis, sepsis, anthrax disease and any combination thereof. In embodiments, for example, the vaccine sample is selected from the group of vaccines consisting of: Pneumococcal; Meningococcal; Haemophilus influenza type B (HIB); anthrax; measles, mumps and rubella (MMR); diphtheria; pertussis and tetanus (DTAP); Diphtheria and tetanus (DT); human papillomavirus (HPV); rotavirus; hepatitis A; hepatitis B.

Any of the systems and methods may also detect exotoxin and/or exotoxin precursors, including exotoxins that may be secreted from bacteria present in the vaccine at some point during the production process or generated by one or more protein precursors from bacteria and then combined to form an exotoxin.

Any of the toxin capture agents may be configured to bind to the toxin that is an exotoxin protein. For example, the at least one toxin capture agent may be a plurality of toxin capture agents configured to bind to a plurality of exotoxin proteins. In an embodiment, the vaccine sample is Anthrax Vaccine Absorbed (AVA). In an embodiment, the vaccine is an Anthrax vaccine, the toxin protein comprises Anthrax lethal factor (LF) and Anthrax edema factor (EF) and the antigen comprises protective antigen (PA).

The provided systems and methods may be further multiplexed to detect both endotoxins and exotoxins in vaccines in which both types of toxin may be present. Further multiplexing allows for determination of endotoxin concentration, exotoxin concentration and vaccine potency, for example, desired immunoactive antigen concentration. In an embodiment, the at least one toxin capture agent comprises: a) an endotoxin capture agent configured to selectively bind to an endotoxin; and b) an exotoxin capture agent configured to selectively bind to an exotoxin protein.

A challenge in multiplexed quantification of toxin concentration and immunoactive antigen concentration is that the immunoactive antigen is typically present within a vaccine sample a much higher concentrations than any toxins. Thus, the provided systems and methods are sensitive and precise, allowing for detection of very low concentration ranges while also robust to allow for accurate quantification of higher concentration of immunoactive antigens. In embodiments, for example, aid immunoactive antigen concentration is selected from the range of 0.1 μg/mL to 10 μg/mL, selected from the range of 0.5 μg/mL to 10 μg/mL, selected from the range of 1 μg/mL to 20 μg/mL, or optionally, greater than or equal to 1 μg/mL. In some embodiments, the toxin concentration is selected from the range of 0.015 μg/mL to 0.1 μg/mL, selected from the range of 0.05 μg/mL to 0.5 μg/mL, less than or equal to 0.1 μg/mL, or optionally, less than or equal to 0.05 μg/mL. In embodiments, the immunoactive antigen concentration is greater than or equal to 3 times the toxin concentration, greater than or equal to 5 times the toxin concentration, or optionally, greater than or equal to 10 times the toxin concentration.

In embodiments, a detection lower limit for the immunoactive antigen is less than or equal to 0.05 μg/mL, less than or equal to 0.1 μg/mL, less than or equal to 0.5 μg/mL, or optionally, less than or equal to 1 μg/mL. In embodiments, a detection lower limit for the toxin is less than or equal to 0.1 μg/mL, less than or equal to 0.05 μg/mL, less than or equal to 0.025 μg/mL, or optionally, less than or equal to 0.015 μg/mL.

Any of the systems and methods described herein may be useful in a vaccine manufacturing process, for example, to monitor changes in antigen or toxin concentration at various stages or for various processes, including the initial to the end steps or processes. The provided methods may also be compatible with vaccine adjuvants. In embodiments, the method is used to optimize one or more vaccine production parameter selected from the group consisting of: bacterial or viral growth condition, bacterial or viral conditions, harvest conditions, one or more purification steps, one or more concentration steps, and any combination thereof. In an embodiment, the vaccine sample comprises an adjuvant, for example, adjuvants containing alum (aluminum salts), squalene, monoglycerides, and fatty acids.

In an embodiment, said substrate has a second antigen capture agent; the step of contacting the substrate forms a plurality of bound complexes further comprising a second antigen capture agent and a second immunoactive antigen bound thereto; and the step of determining the vaccine potency independently determines potency for the immunoactive antigen and the second immunoactive antigen.

In an aspect, provided is a system for determining vaccine potency and toxin concentration: i) a substrate; ii) at least one antigen capture agent bound to the substrate; iii) at least one toxin capture agent bound to the substrate; iv) at least one antigen label agent; and v) at least one toxin label agent; wherein the at least one antigen capture agent is configured to specifically bind an immunoactive antigen and at least one toxin capture agent is configured to specifically bind a toxin when the substrate is contacted with a vaccine sample containing the antigen and the toxin.

In embodiments, the toxin capture agent is configured to bind to one or more endotoxins, one or more exotoxins, or one or more endotoxin and one or more exotoxin. In embodiments, the system further comprises an analyzer, wherein the analyzer comprises: an image capture device; and a processor; wherein the image capture device generates an image of the one or more microarrays, the processor generates a plurality of signals based on the image of the one or more microarrays; the processor compares the plurality of signals to one or more calibration curves and determines an immunoactive antigen concentration and a toxin concentration.

In an aspect, provided is a method for determining vaccine potency and toxin concentration comprising: i) providing a substrate having at least one antigen capture agent, at least one endotoxin capture agent and at least one exotoxin capture agent; ii) contacting the substrate with a vaccine sample to form a plurality of bound complexes, the bound complexes comprising: a) the antigen capture agent and an immunoactive antigen bound thereto, b) the endotoxin capture agent and an endotoxin bound thereto, c) the exotoxin capture agent and an exotoxin bound thereto; and iii) analyzing the substrate to simultaneously determine an immunoactive antigen concentration, an endotoxin concentration and an exotoxin concentration.

In an aspect, provided is a method for quantifying vaccine potency and toxin concentration comprising: a) providing a substrate having a first antigen capture agent, at least one additional antigen capture agent and a toxin capture agent bound to the substrate; b) contacting the substrate with a vaccine sample to form a plurality of bound complexes comprising: i) a first antigen capture agent and a first immunoactive antigen bound thereto; ii) the toxin capture agent and a toxin bound thereto; and iii) at least one additional antigen capture agent and at least one additional immunoactive antigen bound thereto; and c) determining independently vaccine potency for the first immunoactive antigen and each additional immunoactive antigens and determining a toxin concentration for the toxin.

### BRIEF DESCRIPTION OF THE DRAWINGS

FIG. 1A-D illustrates an example of a provided assay. FIG. 1A Nine different mAbs are printed in replicate of 9 on a 16-array microarray slide. FIG. 1B Each array spot comprises epitope-specific mAbs that capture antigen and/or toxin before a label agent (fluorophore-conjugated label Ab) is added to the array (illustration only). The resulting fluorescent images (FIG. 1C) are used to generate a standard curve (FIG. 1D) when an 8-point serial dilution is run on the arrays on the left column of the microarray slide.

FIG. 2A-C shows a platform for determining vaccine potency and toxin concentration. An example system consists of FIG. 2A) modular antigen/toxin-specific reagent kits, FIG. 2B) an imaging system, and FIG. 2C) analysis software.

FIG. 3. shows an illustrative example array layout for 9 total capture agents with each capture agent spotted in a 3×3 matrix of 9 spots.

FIG. 4. shows an illustrative example array layout for 15 total capture agents with each capture agent spotted in a 3×3 matrix of 9 spots.

FIG. 5A provides an example of a toxin capture agent and a toxin label agent bound to a toxin.

FIG. 5B provides an example of an antigen capture agent and an antigen label agent bound to an antigen.

FIG. 6 provides an example of an analyzer including an optical imaging device operably connected to a processor.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Toxin,” refers herein to a substance which is harmful or toxic to humans if present in a sufficient quantity within a vaccine. Toxin may refer to endotoxins, exotoxins and exotoxin precursors (e.g. proteins that may combine with other proteins present in the vaccine sample to form an exotoxin). Toxins may include proteins, lipopolysaccharides, and protein-lipopolysaccharide complexes. Toxins may be generated by bacteria that are specifically designed to be within the vaccine (such as those designed to provide an immune response) or they may be generated by unwanted bacteria or biological matter introduced during the vaccine production process. Toxin, in some embodiments, may exclude inactivated toxins included in vaccines as antigens (for example, diphtheria toxin or tetanus toxoid containing vaccines).

“Antigen” refers to a biological material foreign to a human patient that produces an immune response. In embodiments, antigens may be immunoactive antigens provided in a vaccine to generate an immune response. Antigen may refer to viruses, bacteria, components such as proteins from viruses or bacteria, or other infectious agents in a live, inactivated, or weakened state including exotoxins. Antigen may refer to components of a vaccine specifically included to provoke an immune response.

“Capture agent” as described herein refers to a biologically active agent designed to bind or otherwise interact with specific biological targets, such as antigens, lipopolysaccharides or proteins, including toxins. In some embodiments, capture agents are immobilized on a substrate. Capture agents may specifically bind a desired immunoactive antigen present in a vaccine to create an immune response in a patient in which the vaccine is administered. Capture agents may specifically bind endotoxins, exotoxins and/or exotoxin precursors. In some embodiments, capture agents are monoclonal antibodies.

“Label agent” refers to a biologically active agent designed to bind or otherwise interact with specific biological targets and produce a measurable signal. In some embodiments, label agents are designed to bind or interact with bound complexes formed by capture agents and their targets, but not bind or interact with unbound capture agents allowing for the detection of bound complexes. In embodiments, label agents are fluorescent label agents. In embodiments, label agents comprise a fluorophore coupled to an antibody. In some embodiments, label agents refer to a mixture of fluorophore-coupled antibodies.

“Run-time” refers to the amount of time required to perform as assay, such as determining an immunoactive antigen concentration (potency) and a toxin concentration. In some embodiments, run-time refers to the time required between contacting a sample with a microarray and the time results are provided. Run time may specifically exclude sample preparation time, for example, the time required to dilute or serially dilute a sample due to the presence of adjuvants.

“Detection limit” and “detection lower limit” refer to the lowest concentration in which an assay can accurately detect an analyte. For example, if a concentration is below a detection lower limit, the assay may not be able to detect or to accurately quantify the concentration.

“Quantification limit” and “quantitation lower limit” refer to the lowest concentration in which an assay can accurately quantify an analyte. For example, if a concentration is below a quantitation lower limit, the assay may not be able to accurately quantify the concentration.

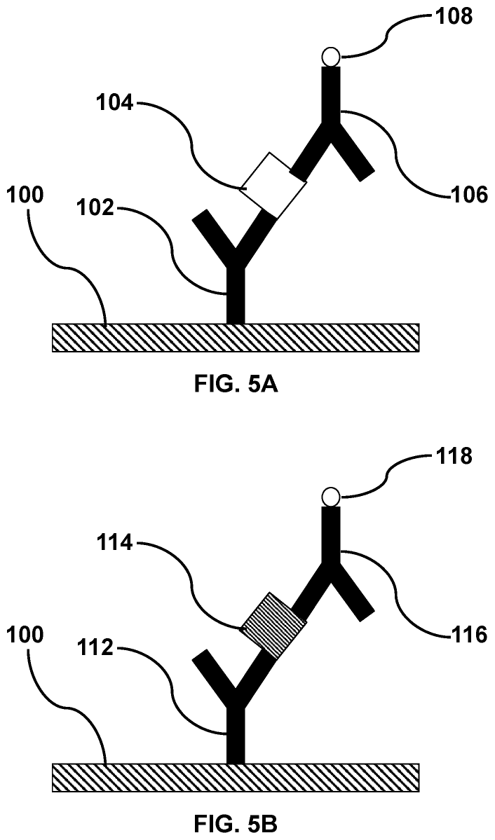

FIG. 5A provides an example toxin capture agent 102 provided on a substrate 100 (e.g. a microarray). The toxin capture agent 102 is designed to interact with or bind to a toxin 104. The toxin labeling agent 106 may then bind to the complex formed by the toxin capture agent 102 and toxin 104. The toxin labeling agent 106 may include a fluorescent label 108 to provide a fluorescent signal for identification or quantification, for example, via imaging or intensity analysis.

Similarly, FIG. 5B provides an example antigen capture agent 112 provided on a substrate 100 (e.g. a microarray). The antigen capture agent 112 is designed to interact with or bind to an antigen 114. The antigen label agent 116 may then bind to the complex formed by the antigen capture agent 112 and antigen 114. The antigen labeling agent 116 may include a fluorescent label 118 to provide a fluorescent signal for identification or quantification, for example, via imaging or intensity analysis.

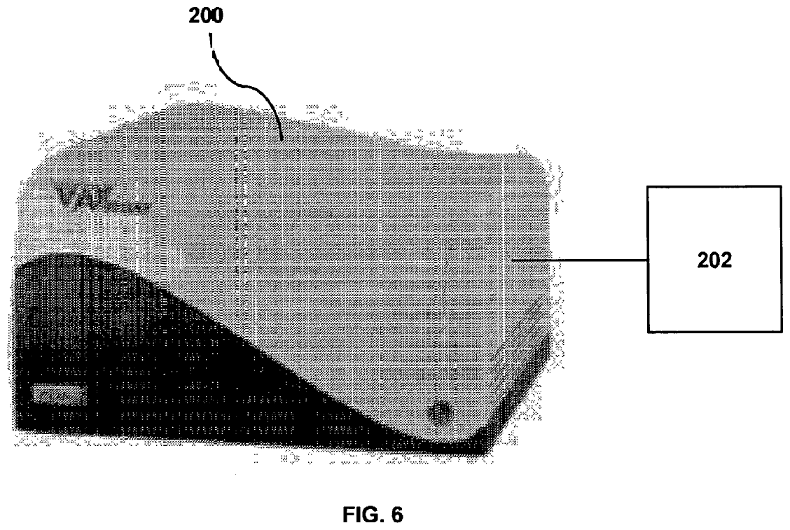

Optionally, an analyzer or image capture device 200 may be included in the systems described herein, as shown in FIG. 6. Image capture devices are known in the art, for example, a digital camera or other device capable of detecting fluorescence or fluorescent intensity. Optionally, the image capture device 200 may be operably connected to a processor 202 (either within the analyzer itself or an external processor such a computer or smartphone) to produce an electronic signal based an image generated by the image capture device 200 and/or analyze the generated image comparing the image or signal to a calibration curve.

### EXAMPLE 1

### Multiplexed Antigen and Toxin Assay System

Described herein is a multiplexed, microarray-based antigen quantification method/kit useful for simultaneously quantifying immune-relevant antigens and potential toxins in vaccines. This technology is relevant for improving both potency assays and measuring safety aspects of vaccine preparations, intermediates, and products.

Purpose. The purpose of the method/kit is to provide an assay that can simultaneously quantify: 1) immune-relevant antigen(s) that comprise a measure of vaccine potency, and 2) relevant toxins that, when present, pose issues to vaccine safety. The toxins being quantified could be exotoxins such as those secreted by a bacterium that can cause damage to the host or components that when present together and able to combine create an exotoxin, endotoxins which are present in many bacterial cell walls and are liberated when the cell dies, or a combination of types of toxin proteins.

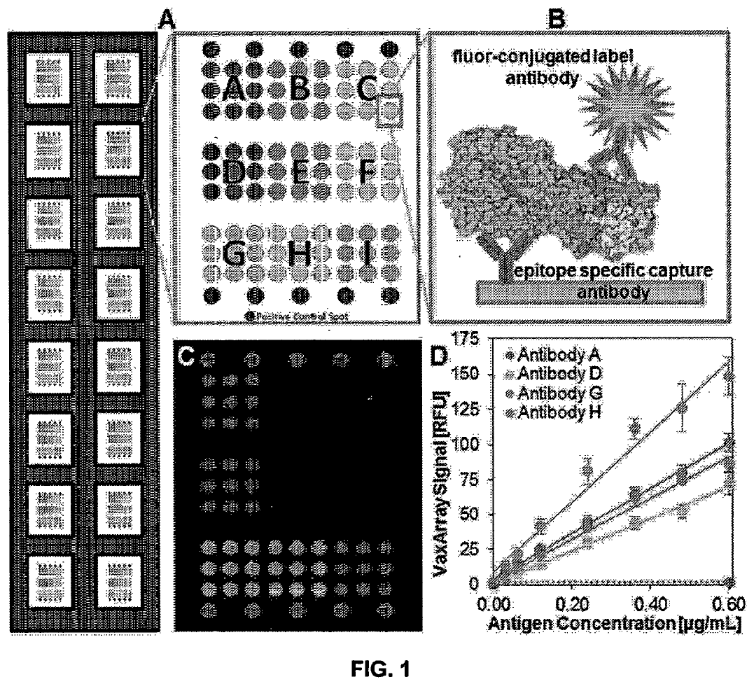

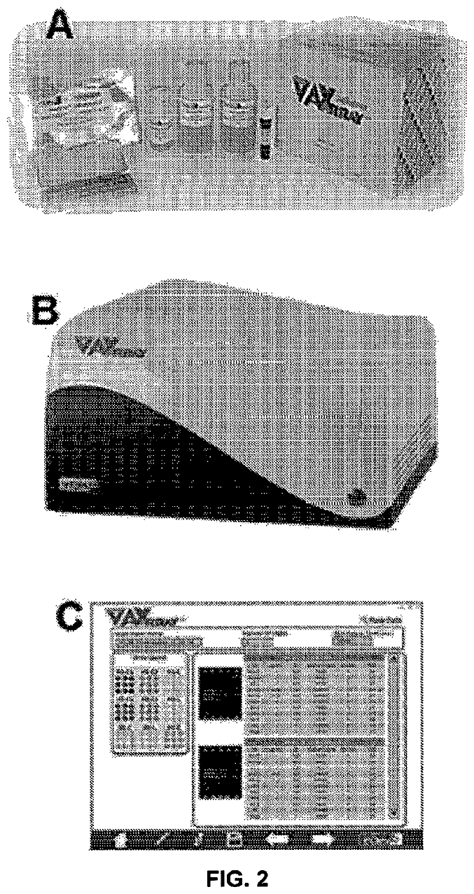

Method. For quantitative analysis, a solution with known antigen and toxin concentrations is serially diluted into a proprietary blocking buffer to generate eight standards with final concentrations that cover the pre-determined linear range of the assay. A vaccine solution of unknown antigen and toxin concentration is prepared by dilution into a proprietary blocking buffer to a final concentration within the predetermined linear range of the assay. The eight standards and the unknown solutions are added to individual, identical microarrays containing at least one antigen capture agent and one toxin capture agent as illustrated in FIG. 1A and incubated for an optimized amount of time. During this time, a complex is formed between the antigen present in the vaccine or standard and the antigen capture agent(s) and a complex is formed between toxin potentially present in vaccine or standard and toxin capture agent. Next, the vaccine and standards solutions are removed from the microarrays and a label agent is added to each microarray. This label agent consists of at least one fluorescently-labeled antigen-specific antibody and at least one fluorescently-labeled toxin-specific antibody. The label agent is incubated with the array for an optimized amount of time. In this time, a complex is formed between the label agent and the antigen-antigen capture agent complex and between the label agent and the toxin-toxin capture agent complex as illustrated in FIG. 1B. The label agent is removed from each microarray and the microarrays are washed in a first buffer solution. The buffer solution is removed, and the microarrays are washed in a secondary wash buffer. The microarrays are then washed with 70% ethanol followed by ultrapure water. The microarrays are then dried by i) using a pipette to remove the remaining water from each array and allowing the slide(s) to air dry or ii) using an air pump or compressor. The washing steps remove any excess antigen, toxin, and label agent. Microarray(s) are subsequently imaged with a fluorescent microarray reader (FIG. 2) optimized for the excitation and emission profiles of the fluorescent labels, the data is extracted by processing the raw microarray image (FIG. 1C), and the fluorescent intensities are reported. Fluorescent intensities from the eight standards are used a plotted against known concentrations for each standard for each antigen-specific capture agent and each toxin-specific capture agent. A line is fit through the resulting data and the equation of the fit line is used as a calibration curve for each capture antibody, antigen and toxin concentrations are calculated for all antigens and toxins (FIG. 1D). The reported florescent intensities for each vaccine solution of unknown concentration are transformed to a concentration in μg/mL using the calibration curve equation.

The described platform consists of three components: i) reagent kits consisting of all required buffers, label reagents, and multiplexed antibody microarray slides, ii) an imaging system, and iii) analysis software designed to enable straightforward compliance with 21 CFR Part 11 and EU Annex 11 (FIG. 2A-C).

### EXAMPLE 2

### Multiplexed, Microarray quantification of Anthrax Vaccine Potency and Toxin Concentration

The examples and descriptions provided below describe anthrax (B. anthracis) as one example of a protein-based vaccine for which quantification of both immune-relevant antigen and secreted toxins (exotoxins) has utility.

Anthrax Vaccine Background—The fatality rate for anthrax by inhalation, a disease caused by Bacillus anthracis, is estimated to be between 45% and 90%, even after using aggressive antibiotic treatment. While the incidence of this infection is nearly nonexistent in humans, the prospective of airborne B. anthracis spores as a bioterrorism agent has prompted the development of an anthrax vaccine stockpile. B. anthracis secretes three proteins known as protective antigen (PA), lethal factor (LF), and edema factor (EF). If the PA protein interacts with LF or EF on the surface of human or animal cells, the resulting toxin can be lethal.

The current method for assessing the potency of anthrax vaccines is a time-consuming, animal-intensive, in vivo potency assay. Using this method, a cohort of guinea pigs is vaccinated and two weeks later injected with a spore suspension of a virulent strain of B. anthracis. Ten days following infection, guinea pigs are observed and deaths are recorded. This method takes 24+ days and costs the lives and distress of an estimated 2,400 guinea pigs per year. In addition to the time and animal consumption of this assay, technicians are put at risk when inoculating live animals with a biosafety level 3 antigen with a reported 50-90% lethality rate. Containment efforts to protect technicians are labor intensive, expensive, and require extensive safety infrastructure.

The in vitro methods and kits described herein may simultaneously replace the current in vivo (animal-based) method for anthrax vaccine potency determination while also reporting concentrations of toxic proteins known to cause adverse reactions to the vaccine if present. This is a dual-purpose assay designed to monitor both anthrax vaccine potency and aspects of product safety by simultaneously quantifying immunogenic and reactogenic toxin components. Specifically for anthrax vaccines, the PA protein is effective for creating an immune response. However, in general the bacterium also generates LF and EF. When LF and EF combine with PA, the complex is a potentially lethal exotoxin. For anthrax vaccine production the relative concentrations of LF and EF are reduced and the concentration of PA is enhanced. However, in the US there are currently no direct measurements of LF and EF. Provided herein are methods and systems to quantify all of these components within the vaccine to help ensure that the vaccine is both potent and safe. The technology allows for reporting antigen concentrations and stability measurements in a range of complex matrices, and could also help manufacturers to optimize bacterial growth and harvest conditions as well as purification steps by enabling quantification of critical proteins throughout the process.

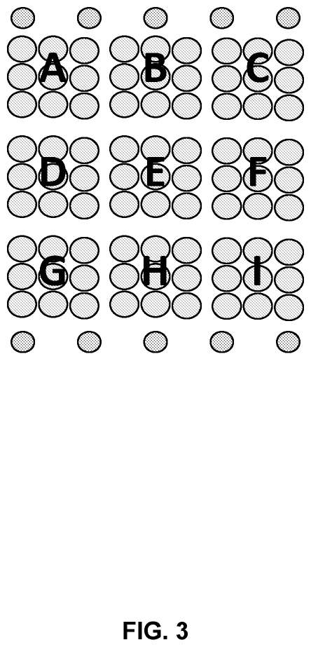

A panel of monoclonal antibodies (mAbs) specifically designed and/or chosen for reactivity and specificity against the proteins of interest, in this case: 1) protective antigen (PA) [the immune-relevant antigen], 2) lethal factor (LF), and edema factor (EF). These mAbs are printed on a microarray slide as illustrated in FIG. 3 where A, B, and C are anti-protective antigen capture agents, D and E are anti-edema factor toxin capture agents, F and G are anti-lethal factor toxin capture agents, and H and I are anti-endotoxin capture agents. The 5 spots in the top and bottom rows are fiducial markers. Vaccine is added to the array and PA, LF, and EF present in the vaccine formulation or intermediate are captured by the respective mAbs. A label agent is then applied to the slide to label the captured proteins for downstream detection. The fluorescent arrays are then imaged using an appropriate imaging-based detection system (for example, the VaxArray® imaging system [InDevR, Inc., Boulder, Colo.]), FIG. 2.

When a dilution series of an appropriate standard material is run by applying different dilutions to each array, a standard curve is generated that can then be used to determine the concentration of PA, LF, and EF in samples of unknown concentration.

In an embodiment, the assay is rapid, with a 2 hour sample to result time. It is simple, safe, and cost effective with no animals, no dangerous bacteria, and no hazardous reagents required. The assay is a stability indicating assay with the ability to monitor antigen stability in anthrax vaccines over time. In some embodiments, the assay is optimized to allow for the quantification of antigen concentration and stability in the presence of adjuvants. The assay may exhibit limits of detection in the ng/mL range which may allow ˜10× dilution of most vaccines, combined with a blocking buffer and the standard use of detergents to solubilize the antigens. In embodiments, detergents are utilized to extract vaccine antigen from aluminum hydrogel, for example, the AVA vaccine to achieve a rapid, animal-free test to simultaneously quantify vaccine potency (PA) and screen for other toxins (LF and EF). The described assay may result in a higher quality vaccine with fewer side effects.

Assay Platform. An illustrative general depiction of the assay is shown in FIG. 1. The unique multiplexed assay platform is outlined and illustrated in FIG. 2.

### EXAMPLE 3

### Pediarix Assay

The examples and descriptions provided below describe Pediarix as one example of a combination protein-based vaccine for which quantification of both immune-relevant antigen and endotoxin has utility.

Pediarix Vaccine Background—Pediarix is a combination vaccine for active immunization against diphtheria, tetanus, pertussis, hepatitis B, and poliomyelitis. Diphtheria is a highly contagious respiratory disease that can cause breathing problems, paralysis, heart failure and death. Tetanus, caused by soil bacterium Clostridium tetani, causes muscle spasms and death if untreated. Pertussis, also known as whooping cough, causes severe coughing spasms and can lead to pneumonia, seizures, brain damage, and death. Hepatitis causes inflammation of the liver and can be life-threatening. Poliomyelitis is a highly infectious viral disease that invades the nervous system and can cause paralysis. Pediarix is approved for use as a three-dose series given as early as 6 weeks of age through 6 years of age.

Pediarix is an example of a combination vaccine that requires expensive and complex in vivo potency methods for each of the 9 vaccine components that make up the vaccine. By testing each component via a different method, several different and unrelated units of measurement are reported for the final formulation. Potency for diphtheria and tetanus vaccine components is determined by measuring the amount of neutralizing antibodies in previously immunized guinea pigs. Pre-formulated preparations of diphtheria toxins and tetanus toxins are tested via the Limit of Flocculation test wherein: a cohort of healthy guinea pigs are vaccinated before sera is collected and mixed with active toxin. Next, the mixture is injected into a cohort of native guinea pigs, and disease systems and time to death is recorded. Pre-formulated vaccine components are compared to reference antigens in the Limit of Flocculation test to produce Lf-units. The potency of the acellular pertussis vaccine components (inactivated PT, FHA, and pertactin) is determined by enzyme-linked immunosorbent assay (ELISA) on sera from previously immunized mice. Briefly, a cohort of healthy mice are vaccinated and sera is collected and analyzed for reactivity with the respective vaccine antigens. An international mouse reference serum containing antibodies to all relevant antigens is provided to monitor consistency of the ELISA testing. Potency is reported in milligrams. Potency of the hepatitis B component is established by an ELISA specific to Hepatitis B antigen (HBsAg) utilizing monoclonal antibodies specific to recombinant HBsAg. Potency is report in milligrams. The potency of the inactivated poliovirus components of Pediarix is determined by using the D-antigen ELISA and by a poliovirus neutralizing cell culture assay on sera from previously immunized rats. Potency for the polio component is reported as D-antigen Units (DU).

While this complex mixture of potency assays remains the standard for final vaccine product release for this and other similar combination vaccines, there are clear cost, time, accuracy, and ethical motivators to move beyond animal testing and the use of a suite of potency tests that are expressed in multiple un-related measurement units.

The methods and kits described herein may simultaneously replace the current suite of potency assays (both in vivo and in vitro) used for Pediarix vaccine and similar combination vaccines with a single unit of measure of potency while also reporting concentrations of endotoxin. This is a unique dual-purpose assay designed to monitor both vaccine potency of combination vaccines and aspects of product safety by simultaneously quantifying all immunogenic and reactogenic toxin components. The technology allows for reporting antigen concentrations and stability measurements in a range of complex matrices, and could help manufacturers to optimize growth and harvest conditions as well as purification steps by enabling quantification of critical proteins throughout the process.

A panel of monoclonal antibodies (mAbs) specifically designed and/or chosen for reactivity and specificity against the proteins of interest are printed in a microarray as shown in FIG. 3 where A is an anti-diphtheria antigen capture agent, B is an anti-tetanus antigen capture agent, C is an anti-polio antigen D capture agent, D is an anti-pertussis toxin antigen capture agent, E is an anti-pertussis filamentous hemagglutinin antigen capture agent, F is an anti-pertussis pertactin antigen capture agent, G is an anti-hepatitis B antigen capture agent, H is an anti-endotoxin capture agent, I is an anti-endotoxin capture agent, and the blue spots are fiducial markers. Vaccine solution is added to the array and diphtheria antigen, tetanus antigen, polio antigen D from each subtype, pertussis toxin antigen, pertussis filamentous hemagglutinin antigen, pertussis pertactin antigen, hepatitis antigen, and any endotoxin present in the vaccine formulation are captured by the respective mAbs. Label agent is then applied to the slide to label the captured antigens and toxins for downstream detection. The fluorescent arrays are then imaged using an appropriate imaging-based detection system (for example, the VaxArray® imaging system [InDevR, Inc., Boulder, Colo.], FIG. 2).

When a dilution series of an appropriate standard material is run by applying different dilutions to each array, a standard curve is generated that can then be used to determine the concentration of each antigen and toxin in samples of unknown concentration.

In an embodiment, the assay is rapid, with a 2 hour sample to result time. It is simple, safe, and cost effective with no animals, no dangerous bacteria, and no hazardous reagents required. The assay is a stability indicating assay with the ability to monitor antigen stability in combination vaccines over time. In some embodiments, the assay is optimized to allow for the quantification of antigen concentration and stability in the presence of adjuvants. The assay may exhibit limits of detection in the ng/mL range which may allow ˜10× dilution of most vaccines, combined with a blocking buffer and the standard use of detergents to solubilize the antigens. In embodiments, detergents are utilized to extract vaccine antigen from aluminum hydroxide, for example, the Pediarix vaccine.

### EXAMPLE 4

### Prevnar-13

The examples and descriptions provided herein describe Prevnar-13 as one example of a multivalent non-protein-based vaccine for which quantification of both immune-relevant antigen and endotoxin has utility.

Prevnar-13 Vaccine Background—Prevnar-13 is a multivalent vaccine for active immunization against Streptococcus pneumoniae serotypes 1, 3, 4, 5, 6A, 6B, 7F, 9V, 14, 18c, 19A, 19F, and 23F. Prevnar-13 is a 13-valent conjugate vaccine containing bacterial polysaccharides conjugated to Diphtheria CRM₁₉₇ protein.

Potency of formulated Prevnar-13 vaccine is determined by quantification of each of the saccharide antigens and by the saccharide to protein ratios in the individual glycoconjugates.

The methods and kits described herein may simultaneously replace the current polysaccharide quantification method for Prevnar-13 vaccine potency determination while also reporting concentrations of endotoxin. This is a dual-purpose assay designed to monitor both multivalent vaccine potency and aspects of product safety by simultaneously quantifying immunogenic and reactogenic toxin components. The technology allows for reporting antigen concentrations and stability measurements in a range of complex matrices and could help manufacturers optimize production conditions as well as purification steps by enabling quantification of critical vaccine components throughout the process.

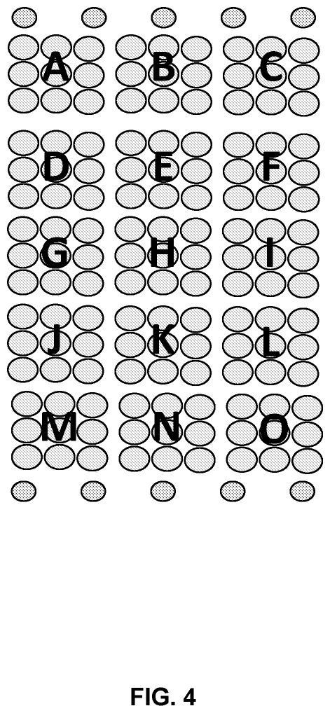

A panel of monoclonal antibodies (mAbs) specifically designed and/or chosen for reactivity and specificity against the bacterial saccharides of interest are printed as shown in FIG. 4 where A through M are anti-capsid polysaccharide antigen capture agents against each of the capsular serotypes within a 13-valent vaccine (1, 3, 4, 5, 6A, 6B, 7F, 9V, 14, 18c, 19A, 19F, and 23F), and N and O are anti-endotoxin capture agents. The 5 spots along the top and bottom rows are fiducial markers.

Vaccine is added to the array, and polysaccharides from each serotype present (1, 3, 4, 5, 6A, 6B, 7F, 9V, 14, 18c, 19A, 19F, and 23F) and any endotoxin present in the vaccine formulation are captured by the respective mAbs. Label agent is then applied to the slide to label the captured antigens and toxins for downstream detection. The fluorescent arrays are then imaged using an appropriate imaging-based detection system (for example, the VaxArray™ imaging system, FIG. 2 [InDevR, Inc., Boulder, Colo.]).

When a dilution series of an appropriate standard material is run by applying different dilutions to each array, a standard curve, also referred to herein as a calibration curve, is generated that can then be used to determine the concentration of each antigen and toxin in samples of unknown concentration.

In an embodiment, the assay is rapid, with a 2 hour or better sample to result time. It is simple, safe, and cost effective with no animals, no dangerous bacteria, and no hazardous reagents required. The assay is a stability indicating assay with the ability to monitor antigen stability in combination vaccines over time. In some embodiments, the assay may be optimized to allow for the quantification of antigen concentration and stability in the presence of adjuvants. The assay may exhibit limits of detection in the ng/mL range which may allow ˜10× dilution of most vaccines, combined with a blocking buffer and the standard use of detergents to solubilize the antigens. In embodiments, detergents are utilized to extract vaccine antigen from aluminum phosphate, for example, the Prevnar-13 vaccine.

### Statements Regarding Incorporation by Reference and Variations

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure. Specific names of compounds or components are intended to be exemplary, as it is known that one of ordinary skill in the art can name the same compounds or components differently.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

Taffs, R. E. Potency Tests of Combination Vaccines. Clinical Infectious Diseases 2001; 33 S362-S366.

Mourez M. Anthrax toxins. Rev. Physiol. Biochem. Pharmacol., Berlin, Heidelberg: Springer Berlin Heidelberg; 2004, p. 135-64. doi:10.1007/s10254-004-0028-2.

BioThrax: How It Works n.d. www.biothrax.com/whatisbiothrax/howitworks.aspx (accessed Oct. 3, 2017).

FDA. Anthrax Vaccine Adsorbed (AVA). Code Fed Regul 1994; 21:chapter 1 (Apr. 1, 1992), subpart C.

Fellows P F, Linscott M K, Ivins B E, Pitt M L, Rossi C A, Gibbs P H, et al. Efficacy of a human anthrax vaccine in guinea pigs, rabbits, and rhesus macaques against challenge by Bacillus anthracis isolates of diverse geographical origin. Vaccine 2001; 19:3241-7.

Stokes W S, Kulpa-Eddy J, Mcfarland R. The International Workshop on Alternative Methods to Reduce, Refine, and Replace the Use of Animals in Vaccine Potency and Safety Testing: introduction and summary. Procedia Vaccinol 2012; 5:1-15. doi:10.1016/j.provac.2011.10.001.

Arciniega J L, Dominguez-Castillo R I. Development and validation of serological methods for human vaccine potency testing: case study of an anthrax vaccine. Procedia Vaccinol 2011; 5:213-20. doi:10.1016/j.provac.2011.10.021.

Little S F, Webster W M, Ivins B E, Fellows P F, Norris S L, Andrews G P. Development of an in vitro-based potency assay for anthrax vaccine. Vaccine 2004; 22:2843-52. doi:10.1016/j.vaccine.2003.12.027.

Ngundi M M, Meade B D, Lin T-L, Tang W-J, Burns D L. Comparison of three anthrax toxin neutralization assays. Clin Vaccine Immunol 2010; 17:895-903. doi:10.1128/CVI.00513-09.

Pombo M, Berthold I, Gingrich E, Jaramillo M, Leef M, Sirota L, et al. Validation of an anti-PA-ELISA for the potency testing of anthrax vaccine in mice. Biologicals 2004;32:157-63. doi:10.1016/j.biologicals.2004.03.002.

Whiting G, Baker M, Rijpkema S. Development of an in Vitro Potency Assay for Anti-anthrax Lethal Toxin Neutralizing Antibodies. Toxins (Basel) 2012; 4:28-41. doi:10.3390/toxins4010028.

Jiménez-Alberto A, Parreiras P, Castelan-Vega J, Sirota L, Arciniega J. Feasibility of the use of ELISA in an immunogenicity-based potency test of anthrax vaccines. Biologicals 2011; 39:236-41. doi:10.1016/j.biologicals.2011.05.001.

Turnbull P C, Broster M G, Carman J A, Manchee R J, Melling J. Development of antibodies to protective antigen and lethal factor components of anthrax toxin in humans and guinea pigs and their relevance to protective immunity. Infect Immun 1986; 52:356-63.

Campbell J D, Clement K H L, Wasserman S S, Donegan S, Chrisley L, Kotloff K L. Safety, reactogenicity and immunogenicity of a recombinant protective antigen anthrax vaccine given to healthy adults. Hum Vaccin n.d.; 3:205-11.

Huang J, Mikszta J A, Ferriter M S, Jiang G, Harvey N G, Dyas B, et al. Intranasal administration of dry powder anthrax vaccine provides protection against lethal aerosol spore challenge. Hum Vaccin n.d.; 3:90-3.

Little S F, Ivins B E, Webster W M, Norris S L W, Andrews G P. Effect of aluminum hydroxide adjuvant and formaldehyde in the formulation of rPA anthrax vaccine. Vaccine 2007; 25:2771-7. doi:10.1016/j.vaccine.2006.12.043.

Cohen-Gihon I, Israeli O, Beth-Din A, Levy H, Cohen O, Shafferman A, et al. Whole-genome sequencing of the nonproteolytic Bacillus anthracis V770-NP1-R strain reveals multiple mutations in peptidase loci. Genome Announc 2014; 2.doi:10.1128/genomeA.00075-14.

Centers for Disease Control and Prevention. Use of anthrax vaccine in the United States: recommendations of the Advisory Committee on Immunization Practices (ACIP). MMWR 2000, 49 n.d.

Zai X, Zhang J, Liu J, Liu J, Li L, Yin Y, et al. Quantitative determination of lethal toxin proteins in culture supernatant of human live anthrax vaccine Bacillus anthracis A16R. Toxins (Basel) 2016; 8:56. doi:10.3390/toxins8030056.

Joellenbeck L M, Zwanziger L L, Durch J S, Strom B L. The Anthrax Vaccine: Is It Safe? Does It Work? National Academies Press (US); 2002. doi:10.17226/10310.

Leppla S H, Robbins J B, Schneerson R, Shiloach J. Development of an improved vaccine for anthrax. J Clin Invest 2002; 110:141-4. doi:10.1172/JC116204.

Geier D A, Geier M R. Anthrax vaccination and joint related adverse reactions in light of biological warfare scenarios. Clin Exp Rheumatol n.d.; 20:217-20.

Pittman P R, Gibbs P H, Cannon T L, Friedlander A M. Anthrax vaccine: short-term safety experience in humans. Vaccine 2001; 20:972-8. doi:10.1016/50264-410X(01)00387-5.

Ready T. US soldiers refuse to fall in line with anthrax vaccination scheme. Nat Med 2004; 10:112-112. doi:10.1038/nm0204-112b.

Turnbull P C B, Broster M G, Carman J A, Manchee R J. Development of antibodies to protective antigen and lethal factor components of anthrax toxin in humans and guinea pigs and their relevance to protective immunity. Infect Immun 1986:356-63.

Kuck L R, Saye S, Loob S, Roth-Eichhorn S, Byrne-Nash R, Rowlen K L. VaxArray assessment of influenza split vaccine potency and stability. Vaccine 2017. doi:10.1016/j.vaccine.2017.02.028.

Zhu D, Huang S, McClellan H, Dai W, Syed N R, Gebregeorgis E, et al. Efficient extraction of vaccines formulated in aluminum hydroxide gel by including surfactants in the extraction buffer. Vaccine 2012; 30:189-94. doi:10.1016/j.vaccine.2011.11.025.

Rinella, Workman, Hermodson, White, Hem. Elutability of proteins from aluminum-containing vaccine adjuvants by treatment with surfactants. J Colloid Interface Sci 1998;197:48-56.

Stoddard R A, Quinn C P, Schiffer J M, Boyer A E, Goldstein J, Bagarozzi D A, et al. Detection of anthrax protective antigen (PA) using europium labeled anti-PA monoclonal antibody and time-resolved fluorescence. J Immunol Methods 2014; 408:78-88. doi:10.1016/j.jim.2014.05.008.

Berthold I, Pombo M-L, Wagner L, Arciniega J L. Immunogenicity in mice of anthrax recombinant protective antigen in the presence of aluminum adjuvants. Vaccine 2005; 23:1993-9. doi: 10.1016/j.vaccine.2004.10.014.

McBride B W, Mogg A, Telfer J L, Lever M S, Miller J, Turnbull P C, et al. Protective efficacy of a recombinant protective antigen against Bacillus anthracis challenge and assessment of immunological markers. Vaccine 1998;16:810-7.

Gorse G, Keitel W, Keyserling H, Taylor D, Lock M, ALVES K, et al. Immunogenicity and tolerance of ascending doses of a recombinant protective antigen (rPA102) anthrax vaccine: A randomized, double-blinded, controlled, multicenter trial. Vaccine 2006; 24:5950-9. doi:10.1016/j.vaccine.2006.05.044.

Ausar S, Hasija L, Li N, Rahman S F. Forced degradation studies: an essential tool for the formulation development of vaccines. Vaccine Dev Ther 2013; Volume 3:11. doi:10.2147/VDT.S41998.

Emergent BioSolutions. BioThrax (Anthrax Vaccine Adsorbed) Product Insert. License No. 1755 n.d.

Kuck L R, Sorensen M, Matthews E, Srivastava I, Cox M M J, Rowlen KL. Titer on chip: New analytical tool for influenza vaccine potency determination. PLoS One 2014; 9:e109616. doi:10.1371/journal.pone.0109616.

Seddon A M, Curnow P, Booth P J. Membrane proteins, lipids and detergents: not just a soap opera. Biochim Biophys Acta—Biomembr 2004;1666:105-17. doi:10.1016/j.bbamem.2004.04.011.

US Patent Publication no. 2012/0316079

International Patent Publication no. WO 2015/18715

We claim:

1. A method for quantifying vaccine potency and toxin concentration comprising:
providing a substrate having at least one antigen capture agent and at least one toxin capture agent;
contacting said substrate with a vaccine sample to form a plurality of bound complexes comprising:
said antigen capture agent and an immunoactive antigen bound thereto; and
said toxin capture agent and a toxin bound thereto; and
determining an immunoactive antigen concentration and a toxin concentration from said plurality of bound complexes, thereby simultaneously quantifying said vaccine potency and toxin concentration.

2. The method of claim 1, wherein said determining step comprises:
generating a plurality of signals from said plurality of bound complexes by contacting said plurality of bound complexes with one or more label agents;
quantifying said plurality of signals to generate a plurality of quantified signals;
providing an antigen calibration curve that defines a relationship between said quantified signals with immunoactive antigen concentration;
providing a toxin calibration curve that defines a relationship between said quantified signals with toxin concentration; and
calculating an immunoactive antigen concentration and a toxin concentration from said plurality of quantified signals, said antigen calibration curve and said toxin calibration curve.

3. The method of claim 2, wherein said label agents comprise fluorescently-labeled antibodies.

4. The method of claim 2 or 3, wherein said one or more label agents comprise an antigen label agent configured to specifically bind to said immunoactive antigen and a toxin label agent configured to specifically bind to said toxin.

5. The method of any of claims 2-4, wherein said step of generating a plurality of signals further comprises imaging said plurality of bound complexes to generate an image, and analyzing said image to generate said plurality of quantified signals.

6. The method of any of claims 2-5, wherein said antigen calibration curve has a linear dynamic range greater than or equal to 1 log.

7. The method of any of claims 2-6, wherein said toxin calibration curve has a linear dynamic range greater than or equal to 1 log.

8. The method of any of claims 1-7, wherein said antigen capture agent and said toxin capture agent are provided in a microarray.

9. The method of claim 8, wherein said substrate supports a plurality of microarrays.

10. The method of claim 9, wherein said plurality of microarrays are each provided in a separate well.

11. The method of any of claims 1-10, wherein said antigen capture agents comprise a panel of monoclonal antibodies.

12. The method of any of claims 1-11, wherein said toxin capture agents comprise a panel of monoclonal antibodies.

13. The method of any of claims 1-12 having a run-time that is less than or equal to 3 hours.

14. The method of any of claims 1-13, wherein said at least one toxin capture agent is configured to selectively bind to said toxin that is an endotoxin.

15. The method of claim 14, wherein said endotoxin is a lipopolysaccharide generated in a bacterial cell wall and is present in intermediate steps in the development of said vaccine sample.

16. The method of claim 14, wherein said endotoxin is a lipopolysaccharide from a gram negative bacterium.

17. The method of claim 16, wherein said gram negative bacterium is selected from the group consisting of: Escherichia coli, Pseudomonas sp., Salmonella typhosa, Salmonella enterica, Klebsiella pneumonia, Serratia marcescens, and Enterobacter sp.

18. The method of any of claims 1-17, wherein said vaccine sample is a vaccine comprising antigen against a disease selected from the group consisting of: influenza virus infection, diphtheria, human papillomavirus (HPV) infection, tetanus, pertussis, poliomyelitis, hepatitis, shingles, varicella, rotavirus infection (gastroenteritis), pneumonia, meningitis, sepsis, anthrax disease and any combination thereof.

19. The method of any of claims 1-18, wherein said vaccine sample is selected from the group of vaccines consisting of: Pneumococcal; Meningococcal; Haemophilus influenza type B (HIB); anthrax; measles, mumps and rubella (MMR); diphtheria, pertussis and tetanus (DTAP); Diphtheria and tetanus (DT); human papillomavirus (HPV); rotavirus; hepatitis A; hepatitis B.

20. The method of any of claims 1-19, wherein said at least one toxin capture agent is configured to bind to said toxin that is an exotoxin protein.

21. The method of claim 20, wherein said at least one toxin capture agent is a plurality of toxin capture agents configured to bind to a plurality of exotoxin proteins.

22. The method of claim 20, wherein said vaccine sample is Anthrax Vaccine Absorbed (AVA).

23. The method any of claims 1-22, wherein said vaccine is an Anthrax vaccine, said toxin protein comprises Anthrax lethal factor (LF) and Anthrax edema factor (EF) and said antigen comprises protective antigen (PA).

24. The method of any of claims 1-23, wherein said at least one toxin capture agent comprises:
an endotoxin capture agent configured to selectively bind to an endotoxin; and
an exotoxin capture agent configured to selectively bind to an exotoxin protein.

25. The method of any of claims 1-24, wherein said immunoactive antigen concentration is selected from the range of 0.1 μg/mL to 10 μg/mL.

26. The method of any of claims 1-25, wherein said toxin concentration is selected from the range of 0.015 μg/mL to 0.1 μg/mL.

27. The method of any of claims 1-26, wherein said immunoactive antigen concentration is greater than or equal to 3 times the toxin concentration.

28. The method of any of claims 1-27, wherein a detection lower limit for said immunoactive antigen is less than or equal to 0.1 μg/mL.

29. The method of any of claims 1-28, wherein a detection lower limit for said toxin is less than or equal to 0.015 μg/mL.

30. The method of any of claims 1-29, wherein said method is used to optimize a vaccine production parameter selected from the group consisting of: bacterial or viral growth condition, bacterial or viral conditions, harvest conditions, one or more purification steps, one or more concentration steps, and any combination thereof.

31. The method of any of claims 1-30, wherein the vaccine sample comprises an adjuvant.

32. The method of claim 31, wherein said adjuvant comprises aluminum hydroxide.

33. The method any of claims 1-32, wherein said substrate has a second antigen capture agent;
said step of contacting said substrate forms a plurality of bound complexes further comprising a second antigen capture agent and a second immunoactive antigen bound thereto; and
said step of determining said vaccine potency independently determines potency for said immunoactive antigen and said second immunoactive antigen.

34. A system for determining vaccine potency and toxin concentration:
a substrate;
at least one antigen capture agent bound to said substrate;
at least one toxin capture agent bound to said substrate;
at least one antigen label agent; and
at least one toxin label agent;
wherein said at least one antigen capture agent is configured to specifically bind an immunoactive antigen and at least one toxin capture agent is configured to specifically bind a toxin when said substrate is contacted with a vaccine sample containing said antigen and said toxin.

35. The system of claim 34, wherein said toxin capture agent is configured to bind to one or more endotoxins, one or more exotoxins, or one or more endotoxin and one or more exotoxin.

36. The system of claim 34 or 35, further comprising an analyzer, wherein said analyzer comprises:
an image capture device; and
a processor;
wherein said image capture device generates an image of said substrate, said processor generates a plurality of signals based on said image of said substrate; said processor compares said plurality of signals to one or more calibration curves and determines an immunoactive antigen concentration and a toxin concentration.

37. A method for determining vaccine potency and toxin concentration comprising:
providing a substrate having at least one antigen capture agent, at least one endotoxin capture agent and at least one exotoxin capture agent bound to said substrate;
contacting said substrate with a vaccine sample to form a plurality of bound complexes, said bound complexes comprising:
said antigen capture agent and an immunoactive antigen bound thereto;
said endotoxin capture agent and an endotoxin bound thereto;
said exotoxin capture agent and an exotoxin bound thereto; and
analyzing said at least one microarray to simultaneously determine an immunoactive antigen concentration, an endotoxin concentration and an exotoxin concentration.

38. A method for quantifying vaccine potency and toxin concentration comprising:
providing a substrate having a first antigen capture agent, at least one additional antigen capture agent and a toxin capture agent bound to said substrate;
contacting said substrate with a vaccine sample to form a plurality of bound complexes comprising:
a first antigen capture agent and a first immunoactive antigen bound thereto;
said toxin capture agent and a toxin bound thereto; and
at least one additional antigen capture agent and at least one additional immunoactive antigen bound thereto; and
determining independently vaccine potency for said first immunoactive antigen and each additional immunoactive antigens and determining a toxin concentration for said toxin.
